(19) United States
(12) Patent Application Publication
(10) Pub. No.: US 2002/0091366 A1
(43) Pub. Date: Jul. 11, 2002

(54) Absorbing structure

(76) Inventor: Christina Abrahamsson, Onsala (SE)

Correspondence Address: Ronald L. Grudziecki, BURNS, DOANE, SWECKER & MATHIS, L.L.P., P.O. Box 1404, Alexandria, VA, 22313-1404, US

(21) Appl. No.: 10/024,201

(22) Filed: Dec. 21, 2001

Related U.S. Application Data
(60) Provisional application No. 60/257,159, filed on Dec. 22, 2000.

(51) Int. Cl. A61F 13/15; A61F 13/20

(52) U.S. Cl. 604/368

(57) ABSTRACT

An absorbing structure with low or non-neutralised superabsorbing polymers. Also disclosed are products having the absorbing structure such as an incontinence garment, feminine sanitary towel, baby diaper, baby pants, panty liners or similar products, as well as the use of low or non-neutralised superabsorbing polymers or the aforementioned absorbing structure for the production of said products.

### CROSS-REFERENCE TO RELATED APPLICATION

This application claims priority under 35 U.S.C. §119(e) to U.S. application Ser. No. 60/257,159 filed on Dec. 22, 2000; the entire content of which is hereby incorporated by reference.

### FIELD OF THE INVENTION

The present invention refers to an absorbing structure having a superabsorbing polymer (SAP) neutralised to a degree of 0-19% and products thereof. The invention also refers to a method for the production of such absorbing structure containing the improved SAP and the use of such low or non-neutralised SAP for the production of an absorbing structure and to products including such absorbing structure.

### BACKGROUND OF THE INVENTION

The use of absorbing structures in different forms of hygiene products is well known. Such hygiene products normally comprise an upper layer through which the liquid is absorbed, an absorbing structure and a lower barrier layer to prevent leakage of the liquid.

To produce high quality absorbing structures, included in absorbing products such as baby diapers, baby pants, incontinence garments, sanitary towels, panty liners and similar products, the absorbing structure is usually mixed with superabsorbing polymers (SAP). Such superabsorbing polymers exist as particles, granules, flakes or fibers.

The superabsorbing polymers are polymers with a high capacity to absorb liquids such as water and body liquids e.g. urine and blood. During absorption, the SAP is swelling to form a non-soluble gel. The absorption capacity of the absorbing structure, and the final product, is dependent on different factors such as where and how the SAP is mixed into the absorbing structure, the shape and the size of the particles, as well as physical and chemical features e.g. absorption rate, gel strength and liquid retaining capacity. The superabsorbing polymers can be mixed or stratified with conventional absorbing material, normally comprising cellulose fibers.

One problem with superabsorbing polymers is how to attach the polymers enough to the fibers in an absorbing structure for keeping them fixed in said structure during transport and usage of the final product. Due to the low efficiency of known methods only a limited amount of SAP can be included in the absorption structure.

SAP is swelling when absorbing liquid and there is a compromise between the amount of SAP included in an absorbing structure and the thickness of the final product not to cause discomfort for the user of the final absorption product.

Different ways of fixing a superabsorbing polymers to the absorbing structure is known. From EP 0 720 488 it is known how to fix the superabsorbing polymers to a fiber structure by the use of thermoplastic polymer fibers to which the superabsorbing polymers are thermically bound. This includes heating the absorbing structure to high temperatures, e.g. 100-300° C., to obtain a good fixing of the SAP to the absorbing structure comprising absorbing material.

Another way of fixing the superabsorbing polymer is described in EP 0 758 220. Herein, the superabsorbing polymer is bound by layering between damped defibrated cellulose pulp or damped tissue. The laminate is compressed between two heated rollers, which will improve the binding of the SAP to the surrounding matrix. This method, however, only keeps the polymers loosely bound to the absorbing structure.

A common feature for both EP 0720 488 and EP 0758 220 is the heating step to high temperatures, e.g. 100-300° C., during fixation of the SAP to the absorbing fibers or structure. Treating the absorbing structure with heat may not always be appropriate and is expensive as well. Also, the superabsorbing polymers are not firmly fixed to the absorbing structure and may get loose during transport or during final usage of the product.

Conventional SAP are polymers, such as polymers of polyacrylic acid. This acid polymer is usually neutralised to a degree of 20-70%, to adjust the pH of the final product. By using NaOH and KOH as neutralisation agents, conversion of the acid—COOH group to a Na or K salt will occur. This conversion will affect the physical behaviour of the SAP, as the polyacrylic acid Na or K salt behaves more hydrophilic than the pure polyacrylic acid. Also, the polymers are hard and brittle due to the salt character of the material.

Another effect of the neutralisation is certain stiffness of the superabsorbing polymer, i.e. less flexibility, of the polymer. Neutralised polymers contain negative charges forcing the polymer to stretch because of electrostatic repulsion forces. This may prevent good contact between the polymer and the surrounding material.

Known in WO 00/35505 is to partially neutralise the SAP to a degree of 20-50% compared to the regular degree of 40-70% in previously known SAP. Apart from a minor increase in stickiness, additional properties gained by partial neutralisation, such as intermediate pH, i.e. pH 4-5, are hampering bacterial growth and bad smell due to the lowered pH.

Still, known methods of fixing SAP to an absorbing structure does not allow a complete fixing of the SAP. Due to this fact, only a limited amount of SAP may be incorporated into the absorbing structure, though this limited amount will thereby decrease the absorption capacity of the final product. To still have a high absorption capacity, the product should be thick which is not desirable to the user.

### SUMMARY

An object of the present invention is to provide an absorbent structure having SAP properly fixed to the fiber structure in the absorbent structure.

Furthermore, it is an object of the invention to provide an absorbing structure comprising low or non-neutralised SAP as well as a method for producing such absorbing structure. Also, the use of SAP neutralised to a degree of 0-19% for the production of a thin, soft and discrete absorbing structure with high absorption capacity and the further use of said absorbing structure for the production of an absorbing product is included.

To fulfil above mentioned objects, an embodiment of the present invention relates to an absorbing structure comprising a SAP neutralised to a degree of 0-19% in order to solve above described problems. A non-neutralised, or alternatively being neutralised to a low degree, SAP is more sticky than a conventional SAP and may, due to the higher stickiness, attach more easily to the fiber matrix to which the SAP is included as an amelioration constituent. By using such low or non-neutralised SAP a higher amount of SAP may be incorporated into an absorbing structure compared to known structures due to an increased stickiness of such low or non-neutralised SAP.

Non-neutralised polyacrylic acid does not have electric charges and are therefore more flexible in contacting the surrounding surfaces. The non-neutralised form of the polymer is, indeed, associated with a high degree of stickiness. Another reason for this stickiness is the possibility to form hydrogen bondings with the polymer network itself and with neighbouring structures. Also contributing to this stickiness of non-neutralised polymers is a possible donation of hydrogen to neighbouring NH₂ groups leading to a leftover anionic charged polymer, which may interact electrostatically with the surrounding structure.

An embodiment of the present invention also relates to a thin, comfortable and discrete product containing an absorbing structure such as an incontinence garment, feminine sanitary towel, baby diaper, baby pants, panty liners or similar products, wherein the absorbing structure of mentioned products comprises the low or non-neutralised SAP described above.

Furthermore, embodiments of the invention include a method for producing such an absorbing structure comprising mixing or layering the absorbing material with the aforementioned SAP having a neutralisation degree of 0-19%. Such an absorbent structure may contain about 60-100% by weight SAP based on the total dry weight of the absorbing structure in the part or the parts where the SAP is distributed.

Embodiments of the invention also include the use of above-mentioned absorbing structure to provide products such as an incontinence garment, feminine sanitary towel, baby diaper, baby pants, panty liners or similar products.

According to an embodiment of the present invention, the absorbent structure can be made thinner than and still be as efficient as previously known alike products due to the incorporation of high amounts of SAP, for absorbing a liquid such as body fluid or blood. The overall thickness of the final product, such as an incontinence garment, feminine sanitary towel, baby diaper, baby pants, panty liners or similar products will also decrease according to the invention which is more comfortable and discrete for the user. Further increasing the comfort and discretion of an absorbing product is hampering of bacterial growth and bad smell when using such a low or non-neutralised SAP, as well as less swelling of the structure during usage.

Ultimately, the present invention will lead to thin products with high absorption capacity and high comfort to the user.

### BRIEF DESCRIPTION OF THE DRAWINGS

FIG. 1 is a schematic view of a diaper comprising SAP.

### DETAILED DESCRIPTION OF THE PREFERRED EMBODIMENTS

As revealed above, an embodiment of the present invention relates to an absorbing structure comprising superabsorbent material with a neutralisation degree of from 0 and up to 19%, products comprising such absorbing structure and a method for production of the mentioned structure. The embodiment helps solve the problem with low amounts of SAP in said absorbing structures by the use of SAP with a higher degree of stickiness due to a low or absent degree of neutralisation of said SAP. Thereby, a thin, discrete and comfortable absorbing structure with a high absorption capacity can be produced which is desirable for the user.

The term “superabsorbing polymer” (SAP) is intended to mean a polymer of cross linked polymers with a capacity to absorb liquid in an amount several times, e.g. ten times or more, their own weight. They further have the capacity to retain absorbed liquid, such as water or body fluid, e.g. blood or urine, also when they are exposed to external pressure. They further have gained wide use in absorbent articles. SAP may be in the format of grains, granules, flakes or fibers, and are usually mixed or layered with other absorption materials such as cellulose fibers. Examples of SAP without limiting the invention are polyacrylic acids such as Casella AB® described in EP 0391 108.

The term “absorbing structure” is intended to include the absorbing part of a product, e.g. diapers, baby pants, incontinence garments, sanitary towels, panty liners or similar products. The absorbing structure may be a mixture of superabsorbing material and “fluff pulp”. Herein, the term “fluff pulp” is intended to mean any type of cellulose fibers included in the absorbing structure.

The term “absorbing capacity” is intended to mean the total capacity to absorb and retain a liquid into the absorbing structure. Included herein are factors influencing the absorbing capacity such as absorption rate as well as diffusion within the absorbing structure both initially and after re-wetting. The amount of additives, such as SAP, as well as the design and shape of the absorbing structure also influence the absorption capacity.

The term “neutralisation” is intended to mean neutralisation of the superabsorbing acidic polymer described above. Depending on the pH of the final product, the SAP is normally neutralised to a degree of 40-70%. NaOH or KOH are used as neutralisation agents. Neutralisation also influences the stiffness of the polymer chains and the stickyness of the polymer network.

The term “conventional SAP” is intended to mean an SAP polymer neutralised to a degree of 20-70%.

As discussed above, the present invention relates to an absorbing structure comprising SAP, the SAP being neutralised to a degree of about 0-19%, preferably to about 0-10%. The low degree or absence of neutralisation effects some of the properties of the absorbing structure in a positive way. One of the major problems with the use of SAP is to attach the SAP properly to the rest of the absorbing structure, normally comprising cellulose fibers. Neutralised SAP is not sticky and is stiff and rigid due to the salt formation with K or Na used in the neutralisation process. Due to such properties, the neutralised SAP is difficult to attach to the rest of the absorbing structure and the addition of high amounts of SAP is difficult.

SAP is used as additives in an absorbing structure and is normally in the form of particles, granules, flakes or fibers. All of them are difficult to attach to the absorbing material in a good way, though flakes and fibers are less difficult due to a larger area per weight unit compared to particles and granules.

The present invention allows, due to the high degree of stickiness in the low and non-neutralised SAP, a higher amount of SAP such as at least about 50%, 60%, 70%, 80%, 90% or about 100% by weight, to be included in the absorbing structure. Normally, there is a compromise between the amount of SAP included in an absorbing structure and the thickness of the final product, which also affects the absorption capacity of the absorbing product wherein the absorption structure is included. Also, since low or non-neutralised SAP have a lower degree of swelling, the risk for gel-blocking is lower. This allows a higher amount of SAP, since they are smaller after swelling compared to conventional SAP, to be included into the same volume of absorption structure.

According to the invention, high amounts of SAP allows a thickness of the final product in dry format to be such as at least about 1, 2, 4, 6 or about 8 mm, which is more comfortable and discreet, both desirable, for the consumer. Also, a less or non-neutralised SAP allows, due to less stiffness of the polymers, the final product to be more soft and flexible affecting the comfort in a positive way for the consumer when using the final product.

The present invention also claims SAP neutralised to a degree of 0-19% as well as an absorbing structure comprising such SAP with or without conventional SAP i.e. SAP with a neutralisation degree of 20-70% in structure.

Less neutralised SAP, i.e. to a degree of about 0-9%, have a lower degree of swelling when absorbing liquids as compared with conventional SAP neutralised to a degree of 20-70%. This means that the absorption capacity, in respect of amount absorbed liquid per time unit, will be lower, due to a slower liquid uptake. Due to less swelling, the risk of blocking the transport of liquid to the rest of the absorption structure surrounding the SAP will decrease. Also, the slow liquid uptake will benefit diffusion of the liquid to other parts of the absorption structure than the immediate contact area to more distant areas. Such diffusion will lead to a more efficient use of the total absorbing structure.

Another aspect of the less or non-neutralised SAP is the pH which will be lower in a less or non-neutralised SAP, about pH 3 in non-neutralised SAP, compared to a conventional SAP. The nature of the chemical bonding in a low or non-neutralised SAP within the polymer structure and to the surrounding area is of a more covalent character for COO⁻ and H⁺, compared to pure ionic bonding due to the salt formation when neutralising the polymer. A low pH will contribute to a low bacterial growth and also hamper bad smell at use of the final product.

In one embodiment, the invention refers to an absorbing structure comprising SAP neutralised to a degree of 0-19%, preferably 0-10%. In the preferred embodiment, the SAP neutralised to a degree of 0-19%, preferably 0-10%, constitutes such as at least about 70, 80, 90 or about 100% by weight SAP, based on the total dry weight of the absorbing structure, in the part or parts where the superabsorbing material is distributed. Specifically, high amounts such as 90, 93, 95, 97, 98 or 100% by weight SAP may be used according to the present invention.

Another embodiment of the invention simply refers to SAP neutralised to a degree of 0-19%, preferably 0-10%.

The absorbing structure according to the present invention may also contain other SAP and absorbing materials, such as fluff pulp, e.g. CTMP i.e., (ChemoThennoMechanical) and/or CP i.e. chemical pulp, synthetic fibers such as for example polypropylene and/or polyethylene fibers, viscose fibers or crosslinked cellulose fibers. The absorbent structure may also contain other superabsorbent material such as for example conventional SAP with a neutralisation degree of 20-70%.

According to the invention, such above described absorption structure is in one embodiment herein a part of or by its own forming an absorbing product such as an incontinence garment, feminine sanitary towel, baby diaper, baby pants, panty liners or similar product described. The said absorption structure may also be used for production of an absorption product such as the above mentioned.

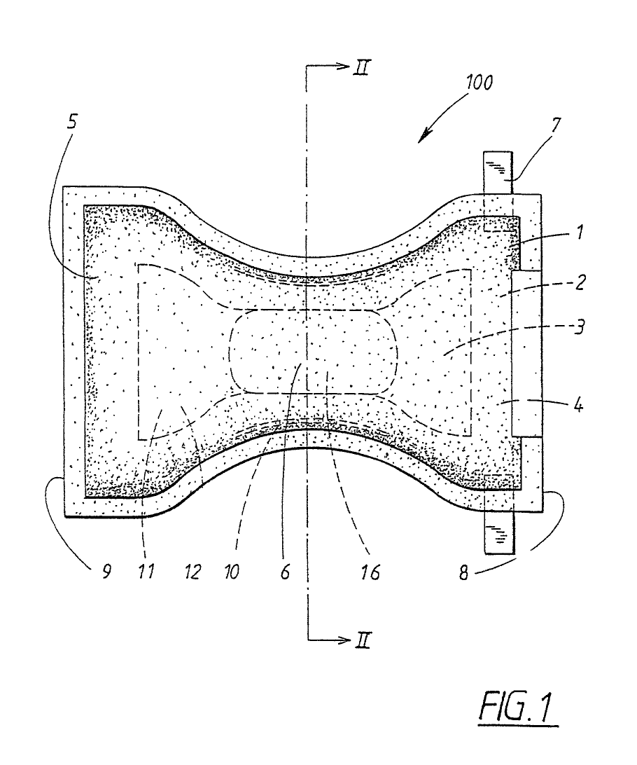

One embodiment of the invention is shown in FIG. 1. The diaper 100 comprises a liquid pervious covering layer 1, made of e.g. fiber cloth or a perforated plastic film, a liquid pervious covering layer 2, made of e.g. plastic film or a hydrophobic fiber cloth, and an absorption structure 3 enclosed between the covering layers 1 and 2.

The diaper is designed to enclose the lower part of a user's body as a pair of absorbing underpants. The design for this purpose is two terminal ends 4, 5 and an in-between located narrower crotch-part 6, which is intended upon usage to be located in the users crotch in-between the users legs. The diaper has edges with a tape 7 near the waistline 8 to form the correct shape upon use. The edges of the tape 7 are affixed to the front of the diaper 5, near the front waist 9, so as to keep the diaper together around the waist of the user.

Also, the diaper contains, according to FIG. 1, pre-stretched elastic parts 10, comprising elastic bands, elastic threads, elastic foam or other, for the purpose, appropriate material. The elastic part IO in FIG. 1 is here shown in a stretched form. As soon as the stretching stops, the elastic parts pull together and form elastic leg-bands on the diaper.

The absorption structure 3 in the diaper shown in FIG. 1 is built up from two layers 11, 12, an upper liquid-absorbing layer 11 and a lower liquid-storage and distribution layer 12. The upper liquid absorbing layer 11 is characterised by a high absorption capacity, e.g. has to absorb large quantities of liquid in a short time, while the lower liquid storage and distribution layer 12 is characterised by a high liquid distribution capacity to be able to drain liquid from the upper liquid absorbing layer 11 and further on distribute the liquid into the lower liquid storage and distribution layer 12. The different characteristics in the two layers 11, 12 may be accomplished by differences in density, wherein a harder compressed fiber structure distribute a liquid better than an equivalent fiber structure with a lower density, the latter one having larger pore-sizes with a higher momentary liquid absorption capacity and a slower liquid distribution capacity.

Differences in absorption capacity between the two layers may also be accomplished by the use of fiber structures with different characteristics, i.e. by the use of a SAP with a different degree of neutralisation, e.g. conventional SAP neutralised to a degree of 20-70% and a SAP according to the present invention. Fluff pulp may also be included wherein the cellulose may be generated chemically e.g. CP or chemi-mechanically e.g. CTMP or may be of synthetic origin such as synthetic fibers such as for example polypropylene and/or polyethylene fibers, viscose fibers or crosslinked cellulose fibers. Cellulose fluff generated chemically has generally a higher liquid distribution capacity then CTMP. Also, fiber structures comprising a cellulose fiber made rigid chemically show a high momentary liquid absorption capacity with a lower liquid distribution capacity than conventional chemical cellulose pulp. Other suitable material to be used in the upper liquid absorbing layer 11, may be a high-loft material of synthetic or natural fibers and/or a fluffy nonwoven material.

In the lower liquid storage and distribution layer 12, of the absorbing structure 3, conventional SAP may be intermixed.

The present invention also includes other embodiments of the absorption structure. The absorption structure may contain both low and non-neutralised SAP, as well as conventional SAP, where both types are equally distributed in the upper and lower layer of the absorption structure. In still another embodiment, the absorbing structure may contain, except from the cellulose fluff, only one type of SAP.

The absorption structure according to the invention includes random mixing of the SAP according to the invention in the absorbing structure as well as layering or stratification of the SAP between tissue layers of cellulose fibers to obtain the most suitable formulation for the specific purpose in an absorbing structure.

Also, the method for production of said absorbing structure is claimed herein. Such a method comprises mixing the absorbing material with SAP with a degree of neutralisation from 0-19%, preferably 0-10%. The SAP may, according to this method, constitute about 60, 70, 80, 90 or about 100% by weight based on the total dry weight of the absorbing structure. Such a method produces a structure that forms a part of an absorbing product such as an incontinence gannent, feminine sanitary towel, baby diaper, baby pants, panty liners or a similar product.

In a specific embodiment, the SAP neutralised to a degree of 0-19%, or preferably 0-10%, is used for the production of an absorbing structure. The SAP herein may constitute such as about 60, 70, 80, 90 or about 100% SAP by weight superabsorbing material based on the total dry weight of the absorbing structure, in the part or the parts where the SAP is distributed.

In a further aspect, the present invention refers to an absorbing product, such as an incontinence garment, feminine sanitary towel, baby diaper, baby pants, panty liners or a similar product, comprising an above described absorbing structure. By such an absorbing structure, it is possible to produce a thin, discrete and comfortable product with a high absorption capacity, which is desirable for the user of the final product.

### EXAMPLES

### Example 1

This example describes, without limiting the invention, an example of a diaper containing SAP neutralised to a degree of 5%. The diaper in the example is shown in FIG. 1.

Overall Design of the Diaper 100

In FIG. 1 is shown a diaper 100. The diaper comprises a liquid pervious covering layer 1, made of a perforated plastic film, a liquid pervious covering layer 2, made of a hydrophobic fiber cloth, and an absorption structure 3 enclosed between the covering layers 1 and 2.

The diaper is designed to enclose the lower part of a user's body as a pair of absorbing underpants. The design for this purpose is two terminal ends 4, 5 and an in-between located narrower crotch-part 6, which is intended to be located upon usage in the users crotch in-between the legs. The diaper has edges with tape 7 near the waistline 8 to form the correct shape upon use. The edges of tape 7 are affixed to the front of the diaper 5, near the front waist 9, so as to keep the diaper together around the waist of the user.

The diaper contains, according to FIG. 1, pre-stretched elastic parts 10, comprising elastic bands, elastic threads, elastic foam or other, for the purpose, appropriate material. The elastic part 10 in FIG. 1 is here shown in a stretched form. As soon as the stretching stops, the elastic parts pull together and form elastic leg-bands on the diaper.

Design of the Absorption Structure

The absorption structure 3 in the diaper is shown in FIG. 1. It is built up from two layers 11, 12 an upper liquid-absorbing layer 11 and a lower liquid storage and distribution layer 12.

Characteristics of the Layers in the Absorption Structure

The different characteristics in the two layers 11, 12, i.e. differences in absorption capacity, is accomplished by the use of fiber structures with different characteristics, i.e. by the use of SAP with different degree of neutralisation, e.g. conventional SAP and SAP according to the invention. The diaper 100 in this example contain SAP neutralised to a degree of 5% in the upper liquid-absorbing layer 11, of the absorbing structure 3. The lower liquid storage and distribution layer 12 contain conventional SAP neutralised to a degree of 60-70%.

The diaper contains 98% by weight SAP based on the total dry weight of the absorbing structure, in the part where the SAP is distributed.

Discussion

The upper liquid-absorbing layer 11 is characterised by a high absorption capacity, e.g. has to absorb large quantities of liquid in a short time, while the lower liquid storage and distribution layer 12 is characterised by a high liquid distribution capacity to be able to drain liquid from the upper liquid-absorbing layer 11 and further on to distribute the liquid in the lower liquid storage and distribution layer 12.

An advantage when placing conventional SAP in the lower liquid storage and distribution layer 12, of the absorbing structure 3, is that a conventional SAP shows a higher total absorption capacity than a low or non-neutralised SAP.

Although this invention has been illustrated and described in accordance with certain preferred embodiments, it is recognized that the scope of this invention is to be determined by the following claims, and equivalents thereof.

What is claimed is:

1. An absorbing structure comprising a superabsorbing polymer having a degree of neutralisation from 0-19%.

2. The absorbing structure according to claim 1, wherein the superabsorbing polymer is neutralised to a degree of from 0-10%.

3. The absorbing structure according to claim 1, wherein the superabsorbing polymer constitutes at least about 60% by weight, based on the total dry weight of the absorbing structure.

4. The absorbing structure according to claim 1, further comprising a conventional superabsorbing polymer with a neutralisation degree of 20-70% in the absorbing structure.

5. The absorbing structure according to claim 1, wherein a thickness of the structure in dry format is at least about 0.5 mm.

6. The absorbing structure according to claim 1, wherein the structure forms part of a product and wherein the product is an incontinence garment, a feminine sanitary towel, a baby diaper, baby pants, or a panty liner.

7. A method for producing an absorbing structure, the absorbing structure including absorbing material and a superabsorbing polymer having a degree of neutralisation from 0-19%, the method comprising: mixing the absorbing material with the superabsorbing polymer and forming the absorbing structure from the absorbing material and the superabsorbing polymer.

8. The method according to claim 7, wherein the superabsorbing polymer constitutes about 60% by weight based on the total dry weight of the absorbing structure.

9. The method according to claim 7, wherein the absorbing structure forms part of a product and the product is an incontinence garment, a feminine sanitary towel, a baby diaper, baby pants, or a panty liner.

10. An absorbing product comprising an absorbing structure as defined in claim 1.

11. The absorbing structure according to claim 1, wherein the superabsorbing polymer constitutes at least about 70% by weight, based on the total dry weight of the absorbing structure.

12. The absorbing structure according to claim 1, wherein the superabsorbing polymer constitutes at least about 80% by weight, based on the total dry weight of the absorbing structure.

13. The absorbing structure according to claim 1, wherein the superabsorbing polymer constitutes at least about 90% by weight, based on the total dry weight of the absorbing structure.

14. The absorbing structure according to claim 1, wherein the superabsorbing polymer constitutes 100% by weight, based on the total dry weight of the absorbing structure.

15. The absorbing structure according to claim 1, wherein a thickness of the structure in dry format is at least about 1 mm.

16. The absorbing structure according to claim 1, wherein a thickness of the structure in dry format is at least about 2 mm.

17. The absorbing structure according to claim 1, wherein a thickness of the structure in dry format is at least about 4 mm.

18. The absorbing structure according to claim 1, wherein a thickness of the structure in dry format is at least about 6 mm.

19. The absorbing structure according to claim 1, wherein a thickness of the structure in dry format is at least about 8 mm.

20. A method for producing an absorbing structure, the absorbing structure including absorbing material and a superabsorbing polymer having a degree of neutralisation from 0-19%, the method comprising: layering the absorbing material with superabsorbing material to produce the absorbing structure.

21. A method according to claim 7, wherein the superabsorbing polymer constitutes about 70% by weight based on the total dry weight of the absorbing structure.

22. The method according to claim 7, wherein the superabsorbing polymer constitutes about 80% by weight based on the total dry weight of the absorbing structure.

23. The method according to claim 7, wherein the superabsorbing polymer constitutes about 90% by weight based on the total dry weight of the absorbing structure.

24. The method according to claim 7, wherein the superabsorbing polymer constitutes about 100% by weight based on the total dry weight of the absorbing structure.

25. The absorbing product according to claim 10, wherein the absorbing product is an incontinence garment, a feminine sanitary towel, a baby diaper, baby pants, or a panty liner.

26. A method according to claim 20, wherein the superabsorbing polymer constitutes about 70% by weight based on the total dry weight of the absorbing structure.

27. The method according to claim 20, wherein the superabsorbing polymer constitutes about 80% by weight based on the total dry weight of the absorbing structure.

28. The method according to claim 20, wherein the superabsorbing polymer constitutes about 90% by weight based on the total dry weight of the absorbing structure.

29. The method according to claim 20, wherein the superabsorbing polymer constitutes about 100% by weight based on the total dry weight of the absorbing structure.
